(19) United States
(12) Patent Application Publication
(10) Pub. No.: US 2017/0253902 A1
(43) Pub. Date: Sep. 7, 2017

(54) Method for Assaying for Loss of an Organism in an Aqueous Liquid

(71) Applicant: Trojan Technologies, London, CA (US)

(72) Inventors: Hugh L. MacIntyre, Dartmouth (CA); John J. Cullen, Barss Comer (CA)

(21) Appl. No.: 15/106,416

(22) PCT Filed: Dec. 15, 2014
(86) PCT No.: PCT/CA2014/000890

§ 371 Date: Jun. 20, 2016

Related U.S. Application Data
(60) Provisional application No. 61/963,982, filed on Dec. 20, 2013.

(51) Int. Cl. C12Q 1/04; G06F 19/00; G01N 21/64; C02F 1/32; B63J 4/00

(52) U.S. Cl. CPC C12Q 1/04; C02F 1/32; B63J 4/002; G01N 21/6428; G06F 19/3406; G01N 2800/7004; G01N 2333/405; C02F 2103/008

(57) ABSTRACT

There is described a method for assaying for loss of viability of a photosynthetic organism (preferably a microorganism) in an aqueous liquid. In a preferred embodiment, the method comprises the step of using fluorescence to correlate the photorepair index for the organism in the aqueous liquid to survivorship of the organism (preferably a microorganism) after it is exposed to ultraviolet radiation, thereby assessing viability.

### CROSS-REFERENCE TO RELATED APPLICATION

The present application claims the benefit under 35 U.S.C. §119(e) of provisional patent application Ser. No. 61/963,982, filed Dec. 20, 2013, the contents of which are hereby incorporated by reference.

### BACKGROUND OF THE INVENTION

Field of the Invention

In a general aspect, the present invention relates to a method for assaying for loss of a target organism (preferably a microorganism) in an aqueous liquid. In another of its aspects, the present invention relates to the use of fluorescence in an assay for loss of organism (preferably microorganism) viability, particularly in an aqueous liquid.

Description of the Prior Art

It is known in the art that aqueous liquids (e.g., municipal wastewater, municipal drinking water, industrial effluents, ballast water on shipping vessels, etc.) can be disinfected of microorganisms using a variety of treatments that lead to immediate or delayed mortality. Treatment with appropriate dosages of ultraviolet radiation (UVR), such as ultraviolet-C (UV-C) radiation, leads to immediate sub-lethal effects resulting in delayed mortality and reproductive impairment. Generally, these effects can only be assessed directly using time-consuming culture-based growth experiments that may take days to months to complete.

Treatment of ballast water on shipping vessels is regulated by the United Nations International Marine Organization (IMO) and the United States Coast Guard (USCG). The USCG has recommended (ETV 2010) that the effectiveness of treatment be assessed using the vital stains fluorescein diacetate (FDA) and 5-chloromethylfluorescein diacetate (CMFDA). Vital stains, therefore, are believed to represent the conventional approach for rapid assessment of ballast water treatment effectiveness.

Fluorescein-based vital stains (e.g., FDA and CMFDA) assay the integrity of the cell membrane and the functionality of esterases in the cells being tested. They have many deficiencies when used to assay viability in phytoplankton. For example, the staining
is time-dependent (Dorsey et al. 1989);
is highly variable between species (Selvin et al. 1988; Murphy and Cowles 1997; Onji et al. 2000; Agusti and Sanchez 2002; Garvey et al. 2007; Peperzak and Brussaard 2011);
varies with growth phase within a species (Gilbert et al. 1992; Garvey et al. 2007); and
can be masked and confounded by green autofluorescence (Tang and Dobbs, 2007; Steinberg et al. 2011) and, in some cases, red chlorophyll autofluorescence (Agusti and Sanchez 2002; Garvey et al. 2007).

Neither cell membranes nor esterases are the primary targets of UVR damage. The primary cause of mortality from UV-C treatment is believed to be through damage to nucleotides (Gieskes and Buma 1997; Sinha and Hader 2002). The damage (e.g., dimerization of the nucleotides in DNA and RNA) interferes with nucleotide replication and transcription for the synthesis of proteins. This damage is not detected by FDA/CMFDA staining.

Consequently, an assay for UV-C-based mortality based on FDA is inaccurate because of a high rate of false positive results: cells that are successfully treated and incapable of reproduction can retain intact membranes and functional esterases and thus stain heavily with FDA. Although incapable of growth and reproduction (functionally non-viable in the natural environment), they are assessed as being healthy—see FIG. 1.

The purpose of ballast water treatment is to prevent the introduction of potentially invasive microorganisms; this can be accomplished by killing them or making them non-viable, i.e., incapable of reproduction and thus unable to colonize receiving waters.

Treatment with UVR, a proven technology for municipal drinking water and municipal wastewater disinfection has been described for ballast water applications—see, for example, International Patent Publication Number WO 2010/130031 [Fraser] and International Patent Publication Number WO 2012/061924 [DaCosta et al.]. However, since UVR acts primarily through reproductive impairment and delayed mortality rather than through immediate killing, assessment of the technology for microorganisms is difficult. Broadly, this is for two reasons:
Techniques for measuring reproduction directly are time-consuming and subject to uncertainty when applied to mixed assemblages in natural samples—The direct measure of a microorganism's viability is the ability to reproduce, i.e., grow in culture (as determined with so-called grow-out or regrowth methods, e.g., Liebich et al. 2012). In principle, culture-based methods such as the Most Probable Numbers (MPN) technique provide the gold-standard assessment of viability (cf. Throndsen 1978). They require days to months to perform, though, and are thereby unsuitable for routine verification of ballast water treatment compliance of a given shipping vessel. Further, when applied on naturally-occurring plankton assemblages, uncertainties are introduced because some aquatic microorganisms (including heterotrophs for which culture conditions are not designed) cannot be cultured reliably. Therefore, the effects of UVR on their viability cannot be reliably measured directly using MPN.
The mode of action of UVR differs from those of other disinfection technologies, so commonly used “live vs. dead” assays greatly underestimate the effectiveness of UVR in preventing the introduction of invasive microorganisms—Damage to DNA (e.g., formation of pyrimidine dimers) is the principal reason why UVR treatment inactivates microbes (Gieskes and Buma 1997; Sinha and Hader 2002). Cells that have been rendered nonviable by UVR treatment can retain some metabolic function and thereby appear as living when assessed with vital stains that are currently used to measure the effectiveness of ballast water treatment (ETV 2010)—see FIG. 1. Consequently, ballast water treatment guidelines that are based on validation with vital stains—i.e., “living” as determined with vital stains, rather than “viable”, defined as the ability to reproduce can impose inappropriately high design doses that would result in the need for larger treatment systems with consequential greater energy demand.

In light of the above, reliable and rapid alternatives to culture-based assays are needed, but existing approaches based on vital stains do not reliably detect delayed mortality and reproductive impairment from UVR treatment. Assessments of damage to photosynthetic systems, based on measurements of chlorophyll fluorescence, can detect damage due to UVR, but measures of photodamage alone do not reliably indicate mortality or reproductive impairment, in part because the molecular targets associated with damage to photosystems are not the same as for DNA replication and protein synthesis.

Therefore, there is a pressing need for a rapid assay of more general metabolic damage. Preferably, such an assay would correlate with reproductive impairment as determined through culture-based assays of viability.

### SUMMARY OF THE INVENTION

It is an object of the present invention to obviate or mitigate at least one of the above-mentioned disadvantages of the prior art.

It is another object of the present invention to provide a novel approach to assaying for loss of viability of an organism in an aqueous liquid.

Accordingly, in one of its aspects, the present invention provides use of fluorescence in an assay for loss of viability of an organism in an aqueous liquid after the organism as been exposed to a stressor, the use comprising assessing the ability of the organism to undergo photorepair.

In another of its aspects, the present invention provides the use of fluorescence in an assay for loss of viability of an organism in an aqueous liquid after the organism as been exposed to radiation, the use comprising assessing the ability of the organism to undergo photorepair.

In yet another of its aspects, the present invention provides the use of fluorescence in an assay for loss of viability of an organism in an aqueous liquid after the organism as been exposed to ultraviolet radiation, the use comprising assessing the ability of the organism to undergo photorepair.

In yet another of its aspects, the present invention provides the use of variable fluorescence in an assay for loss of viability of an organism in an aqueous liquid after the organism as been exposed to a stressor, the use comprising assessing the ability of the organism to undergo photorepair.

In yet another of its aspects, the present invention provides the use of variable fluorescence in an assay for loss of viability of an organism in an aqueous liquid after the organism as been exposed to radiation, the use comprising assessing the ability of the organism to undergo photorepair.

In yet another of its aspects, the present invention provides the use of variable fluorescence in an assay for loss of viability of an organism in an aqueous liquid after the organism as been exposed to ultraviolet radiation, the use comprising assessing the ability of the organism to undergo photorepair.

In yet another of its aspects, the present invention provides a method for assaying for loss of viability of an organism in an aqueous liquid after the organism has been exposed to a stressor, the method comprising the step of assessing the ability of the organism to undergo photorepair.

In yet another of its aspects, the present invention provides a method for assaying for loss of viability of an organism comprised in an aqueous liquid after the organism has been exposed to a stressor, the method comprising the steps of:

(a) measuring the variable fluorescence (Fᵥ) of an untreated sample of the organism prior to exposure to the stressor;

(b) measuring the variable fluorescence (Fᵥ) of a treated sample of the organism after exposure to the stressor;

(c) calculating a photorepair index using the measurements obtained in Steps (a) and (b); and

(d) correlating the photorepair index calculated in Step (c) to a normalized viability for the organism.

In yet another of its aspects, the present invention provides a system for assaying loss of viability of an organism in an aqueous liquid, the system comprising:

(a) a sample housing for receiving a sample of the aqueous liquid;

(b) a device configured to measure the fluorescence of the organism in the aqueous liquid; and

(c) a computer element configured to correlate the photorepair index for the organism in the aqueous liquid to survivorship of the organism after the organism has been exposed to a stressor.

The present inventors have discovered that measurements of damage to the photosynthesis repair process serve as good proxies for generalized metabolic impairment and the loss of viability of an organism, preferably a microorganism. Thus, the present inventors have developed a rapid assay of damage to photosynthetic systems, and repair of that damage, preferably based on measurements of fluorescence, preferably variable fluorescence, most preferably variable chlorophyll fluorescence. The present inventors have established that an index based on such measurements can be used to reliably predicted survivorship in photosynthetic microorganisms treated with UV-C. By extending fluorescence-based assays of photodamage to quantify both damage to photosystems and its repair (which is dependent on protein synthesis), a rapid and sensitive assessment of general metabolic impairment and loss of viability has been been developed. This represents an improvement over the above-mentioned prior art approach of assessment of damage to photosynthesis, which is not as reliable an indicator of the loss of viability after UV-C treatment as the present assay.

From a general perspective, damage to the photosynthesis repair process of the organism is assessed after subjecting the organism to a so-called stressor. As used throughout this specification, the term “stressor” has a broad meaning and is intended to encompass an agent, condition or other stimulus that causes stress to the organism. In a preferred embodiment, the stressor is is selected from the group consisting of: exposure to a chemical, exposure to mechanical energy, thermal shock, dark storage and any combination thereof In another preferred embodiment, the stressor is ultraviolet radiation such as UV-C radiation.

Thus, in a preferred embodiment, the present invention relates to a protocol of fluorescence measurements during manipulation of the ambient light field after UV-C treatment of an aqueous liquid containing the organism. This preferred embodiment relates to a method for rapid assessment of metabolic impairment in photosynthetic organisms that is significantly more accurate as a measure of loss of viability than methods based on vital stains or on the direct determination of fluorescence parameters alone.

The invention thus relates a rapid and reliable method for assessing the loss of viability in photosynthetic organisms (preferably microorganisms) that may be advantageously used in the evaulation of disinfection treatments or other stresses placed on such organisms.

### BRIEF DESCRIPTION OF THE DRAWINGS

Embodiments of the present invention will be described with reference to the accompanying drawings, wherein like reference numerals denote like parts, and in which:

FIG. 1 illustrates a comparison of UV-C-induced mortality (as log₁₀ reduction in viable cell number) in three microalgal cultures, Thalassiosira weissflogii, Heterosigma akashiwo and Isochrysis galbana, estimated by culture-based experiments and by staining with FDA;

FIG. 2 illustrates dose-response curves for three microalgal cultures, Thalassiosira weissflogii, Heterosigma akashiwo and Isochrysis galbana and relationships between viability determined from MPN experiments and the variable fluorescence parameter, Fᵥ, measured after treatment;

FIG. 3 illustrates dose-response curves for three microalgal cultures, Thalassiosira weissflogii, Heterosigma akashiwo and Isochrysis galbana (left) and relationships between viability determined from MPN experiments and a Photorepair Index (PRI), measured immediately after treatment (right);

FIG. 4 illustrates an example of determination of the input parameters for PRI based on sequential incubations at high and low light intensities—Fᵥ is measured on a sample prior to (Untreated) and after (Treated) treatment with UVR (in each case, a dark-acclimated sample is exposed to high light and a successive period of low light, during which Fᵥ is measured);

FIG. 5 illustrates an example of determination of the input parameters for PRI based on parallel incubations at high light with and without a chloroplastic protein synthesis inhibitor—in this case, the antibiotic lincomycin was used as an inhibitor (Fᵥ is measured on a sample prior to (Untreated) and after (Treated) treatment with UVR and, in each case, a dark-acclimated sample is exposed to high light in parallel incubations with and without the protein synthesis inhibitor);

to FIG. 6 illustrates a schematic of implementation of a first embodiment of the present method; and

FIG. 7 illustrates a schematic of implementation of a second embodiment of the present method.

### DETAILED DESCRIPTION OF THE PREFERRED EMBODIMENTS

The present invention relates to the follow independent uses:
use of fluorescence in an assay for loss of viability of an organism in an aqueous liquid after the organism as been exposed to a stressor, the use comprising assessing the ability of the organism to undergo photorepair;
use of fluorescence in an assay for loss of viability of an organism in an aqueous liquid after the organism as been exposed to radiation, the use comprising assessing the ability of the organism to undergo photorepair;
use of fluorescence in an assay for loss of viability of an organism in an aqueous liquid after the organism as been exposed to ultraviolet radiation, the use comprising assessing the ability of the organism to undergo photorepair;
use of variable fluorescence in an assay for loss of viability of an organism in an aqueous liquid after the organism as been exposed to a stressor, the use comprising assessing the ability of the organism to undergo photorepair;
use of variable fluorescence in an assay for loss of viability of an organism in an aqueous liquid after the organism as been exposed to radiation, the use comprising assessing the ability of the organism to undergo photorepair; and
use of variable fluorescence in an assay for loss of viability of an organism in an aqueous liquid after the organism as been exposed to ultraviolet radiation, the use comprising assessing the ability of the organism to undergo photorepair.

Preferred embodiments of these uses may include any one or a combination of any two or more of any of the following features:
the stressor is selected from the group consisting of: exposure to a chemical, exposure to mechanical energy, thermal shock, dark storage and any combination thereof;
the radiation is UV-C radiation;
the ultraviolet radiation has a wavelength in the range of from about 100 nm to about 280 nm;
the organism is a microorganism;
the aqueous liquid is water; and/or
the aqueous liquid is ballast water from a shipping vessel.

In another of its aspects, the present invention relates to a method for assaying for loss of viability of an organism in an aqueous liquid after the organism has been exposed to a stressor, the method comprising the step of assessing the ability of the organism to undergo photorepair. Preferred embodiments of these use may include any one or a combination of any two or more of any of the following features:
the stressor is selected from the group consisting of: exposure to a chemical, exposure to mechanical energy, thermal shock, dark storage and any combination thereof;
the stressor is ultraviolet radiation;
the stressor is UV-C radiation;
the stressor is ultraviolet radiation having a wavelength in the range of from about 100 nm to about 280 nm;
the assessing step comprises conducting a fluorescence test on the organism;
the assessing step comprises conducting a variable fluorescence test on the organism;
the organism is a microorganism;
the aqueous liquid is water; and/or
the aqueous liquid is ballast water from a shipping vessel.

In another of its aspects, the present invention relates to a method for assaying for loss of viability of an organism in an aqueous liquid after the organism has been exposed to a stressor, the method comprising the step of correlating the photorepair index for the organism in the aqueous liquid to survivorship of the organism after the organism has been exposed to the stressor. Preferred embodiments of these uses may include any one or a combination of any two or more of any of the following features:
the stressor is selected from the group consisting of: exposure to a chemical, exposure to mechanical energy, thermal shock, dark storage and any combination thereof;
the stressor is ultraviolet radiation;
the stressor is UV-C radiation; v the stressor is ultraviolet radiation having a wavelength in the range of from about 100 nm to about 280 nm;
the photorepair index is calculated by subjecting the organism to a fluorescence test;
the photorepair index is calculated by subjecting the organism to a variable fluorescence test;
the organism is a microorganism;
the aqueous liquid is water; and/or
the aqueous liquid is ballast water from a shipping vessel.

In another of its aspects, the present invention relates to method for assaying for loss of viability of an organism comprised in an aqueous liquid after the organism has been exposed to a stressor, the method comprising the steps of: (a) measuring the variable fluorescence (Fᵥ) of an untreated sample of the organism prior to exposure to the stressor; (b) measuring the variable fluorescence (Fᵥ) of a treated sample of the organism after exposure to the stressor; (c) calculating a photorepair index using the measurements obtained in Steps (a) and (b); and (d) correlating the photorepair index calculated in Step (c) to a normalized viability for the organism. Preferred embodiments of these use may include any one or a combination of any two or more of any of the following features:
Step (a) comprises one or more of the following: (i) measuring the variable fluorescence (Fᵥ) of the untreated sample of the organism prior to exposure to radiation to obtain Fᵥ(0)^{Untreated}; (ii) incubating the untreated sample in the presence of radiation at a first intensity to induce a prescribed reduction in Fᵥ from Fᵥ(0)^{Untreated}; (iii) measuring the variable fluorescence (Fᵥ) of the untreated sample after Step (ii) to obtain Fᵥ(1)^{Untreated}; (iv) incubating the untreated sample in presence of radiation at a second intensity to induce photorepair and an increase in Fᵥ from Fᵥ(1)^{Untreated}; (v) measuring the variable fluorescence of the untreated sample after Step (iv) to obtain Fᵥ(2)^{Untreated};
Step (ii) is conducted for a period of from about 10 minutes to about 120 minutes;
the first intensity is in the range of from about 2 μmol photons m⁻²s⁻¹ to about 4000 μmol photons m⁻²s⁻¹;
Step (ii) comprises incubating the untreated sample in presence of photosynthetically active radiation;
Step (ii) comprises incubating the untreated sample in presence of photosynthetically active radiation having one or more wavelengths substantially within the range of from about 400 nm to about 700 nm;
the prescribed reduction in Fᵥ in Step (ii) is in the range of from about 20% to 100%;
the prescribed reduction in Fᵥ in Step (ii) is in the range of from about 30% to 80%;
the prescribed reduction in Fᵥ in Step (ii) is in the range of from about 35% to 55%;
Step (iv) is conducted for a period of from about 30 minutes to about 240 minutes;
the second intensity is in the range of from about 1 μmol photons m⁻²s⁻¹ to about 100 μmol photons m⁻²s⁻¹;
the second intensity is in the range of from about 10 μmol photons m⁻²s⁻¹ to about 50 μmol photons m⁻²s⁻¹;
Step (iv) comprises incubating the untreated sample in presence of photosynthetically active radiation having wavelengths from about 400 nm to about 700 nm;
Step (iv) comprises incubating the untreated sample in presence of photosynthetically active radiation having one or more wavelengths substantially within the range of from about 400 nm to about 700 nm; substantially the same radiation is used Step (ii) and Step (iv);
Step (b) comprises one or more of the following: (i) incubating the treated sample in presence of photosynthetically active radiation at a first intensity to induce a prescribed reduction in Fᵥ from Fᵥ(0)^{Untreated}; (ii) measuring the variable fluorescence of the treated sample after Step (ii) to obtain Fᵥ(1)^{Treated}; (iii) incubating the untreated sample in presence of radiation at a second intensity to induce photorepair and an increase in Fᵥ from Fᵥ(1)^{Treated}; and (iv) measuring the variable fluorescence of the treated sample after Step (iii) to obtain Fᵥ(2)^{Treated};
Step (i) is conducted for a period of from about 10 minutes to about 120 minutes;
the first intensity is in the range of from about 2 μmol photons m⁻²s⁻¹ to about 4000 μmol photons m⁻²s⁻¹;
Step (i) comprises incubating the untreated sample in presence of photosynthetically active radiation having wavelengths from about 400 nm to about 700 nm;
Step (i) comprises incubating the untreated sample in presence of photosynthetically active radiation having one or more wavelengths substantially within the range of from about 400 nm to about 700 nm;
Step (iii) is conducted for a period of from about 30 minutes to about 240 minutes;
the second intensity is in the range of from about 1 μmol photons m⁻²s⁻¹ to about 100 μmol photons m⁻²s⁻¹;
the second intensity is in the range of from about 10 μmol photons m⁻²s⁻¹ to about 50 μmol photons m⁻²s⁻¹;
Step (iii) comprises incubating the untreated sample in presence of photosynthetically active radiation having wavelengths from about 400 nm to about 700 nm;
Step (iii) comprises incubating the untreated sample in presence of photosynthetically active radiation having one or more wavelengths substantially within the range of from about 400 nm to about 700 nm;
substantially the same radiation is used Step (i) and Step (iii);
Step (c) comprises calculating the photorepair index using one of the following equations:

$$\begin{matrix}PRI = \frac{{[ {F}_{v} ( 2 ) - {F}_{v} ( 1 ) ]}^{Treated}}{{[ {F}_{v} ( 0 ) ]}^{Untreated}} \\ or & ( Eq . 1 ) \\ PRI = \frac{{[ {F}_{v} ( 2 ) - {F}_{v} ( 1 ) ]}^{Treated}}{{[ {F}_{v} ( 2 ) - {F}_{v} ( 1 ) ]}^{Untreated}} & ( Eq . 2 ) \\ or & \\ PRI = \frac{{[ {F}_{v} ( 2 ) - {F}_{v} ( 1 ) ]}^{Treated}}{{{F}_{v} ( 1 )}^{Untreated}} ; & ( Eq . 3 )\end{matrix}$$

the organism is a microorganism;
the aqueous liquid is water; and/or
the aqueous liquid is ballast water from a shipping vessel.

In one of its aspects, the present invention relates to the use of fluorescence, preferably variable fluorescence, more preferably variable chlorophyll fluorescence, in an assay for loss of organism (preferably microorganism) viability in an aqueous liquid.

Damage to photosynthetic systems can be detected rapidly with sensitive assays of variable chlorophyll fluorescence, Fᵥ. This can be achieved, for example, using a fluorometer with modulated excitation, including for example, pump-and-probe, PAM, FRRF or FIRe fluorometers (e.g., Genty et al. 1989; Schreiber et al. 1995; Gorbunov and Falkowski 2004). Fᵥ can also be assessed using a fluorometer with a constant excitation when used in conjunction with an electron transport inhibitor such as 3-(3,4-dichlorophenyl)-1,1-dimethylurea (Cullen et al. 1986; Vincent 1980).

Although suggested as means of assessing UV-C-based damage (First and Drake 2013b), Fᵥ alone is not a robust proxy for delayed mortality nor the impairment of reproductive ability in photosynthetic microorganisms. The primary molecular targets for damage to photosynthetic systems are not the same as for UV-C-killing and there is likely to be significant variability between species and growth conditions in the relationship between fluorescence changes and loss of viability (e.g., Campbell et al. 1998; Xiong 2001; Bouchard et al. 2005; Bouchard et al. 2008; Key et al. 2010). The failure of Fᵥ alone to reliably predict UV-C-based mortality is illustrated in FIG. 2.

Damage to photosynthetic systems by ultraviolet radiation (UVR) is countered by repair mechanisms. These involve protein synthesis and both mitigate damage during exposure and restore photosynthetic competence after the exposure if the cells are exposed to visible light (Vasilikiotis and Melis 1994; Neidhardt et al. 1998; Adir et al. 2005). The repair mechanisms are thus among the primary targets for UVR-induced damage and mortality; damage to photosynthetic repair mechanisms from UV-C happens at the same time as the more generalized damage to nucleotides that leads to metabolic impairment and the loss of reproductive ability. The present inventors have discovered that measurements of damage to the photosynthesis repair process serve as good proxies for generalized metabolic impairment and the loss of viability.

The present inventors have developed a rapid assay of damage to photosynthetic systems, and repair of that damage, based on measurements of chlorophyll fluorescence. As will be further discussed below, the present inventors have demonstrated that an index based on these measurements can be used to reliably predict survivorship in photosynthetic microorganisms treated with UV-C. In this regard, prior art assessments of damage due to photosynthesis alone were found by the present inventors not to be reliable indicators of the loss of viability after UV-C treatment.

Thus, a protocol of fluorescence measurements during manipulation of the ambient light field after UV-C treatments has been developed by the present inventors. In a preferred embodiment, it is a method for rapid assessment of metabolic impairment in photosynthetic organisms that is significantly more accurate as a measure of loss of viability than methods based on vital stains or on the direct determination of fluorescence parameters alone.

Variable chorophyll a fluorescence, Fᵥ (i.e., maximal fluorescence, Fₘ, minus initial fluorescence, Fₒ) is preferably measured using either a fluorometer with modulated excitation such as a pump-and-probe, pulse amplitude modulated (PAM) fluorometry, fast repetition rate fluorometry (FRRF), fluorescence induction and relaxation (FIRe), etc., or with a fluorometer with a stable excitation intensity in conjunction with an electron transport inhibitor such as 3-(3,4-dichlorophenyl)-1,1-dimethylurea. Data collection protocols are described by the manufacturer of the particular instrument used.

To optimize the accuracy of measurement of Fᵥ, it is preferred that each sample be subjected to a period of dark acclimation sufficient to restore photochemical quenching before each measurement of fluorescence.

Preferably, time-dependent changes in Fᵥ are assessed for an untreated sample and for a sample or samples subjected to stress.

In preferred embodiment, the stress is in the form of exposure to UV-C radiation this is shown schematically in FIG. 6.

Preferably, both the untreated sample and the treated sample(s) are subjected to the same experimental protocol. Preferably, all samples are maintained at temperatures corresponding to conditions in their parent populations (i.e., the temperature in the water body from which they are collected).

Preferably, Fᵥ is measured over the course of an assessment protocol under the following consecutive irradiance conditions.

First, a sample is dark-acclimated and Fᵥ is measured prior to illumination. In the case of the untreated sample, this is Fᵥ(0)^{Untreated}.

The sample is then incubated, preferably in visible light, at an irradiance high enough and for a period long enough to induce a prescribed or pre-determined reduction (e.g., 50%) in Fᵥ in the untreated sample. For example, the sample can be exposed for a period of from about 10 minutes to about 120 minutes to photosynthetically active radiation (PAR) having an intensity of from about 2 μmol photons m⁻²s⁻¹ to about 4000 μmol photons m⁻²s⁻¹. Preferably, the irradiance is dominated by wavelengths of from about 400 nm to about 700 nm. Alternately, UV-B and UV-A radiation (about 280 nm to 400 nm) can be applied. Non-limiting examples of suitable radiation sources for this purpose may be selected from xenon lamps, quartz halogen lamps, fluorescent lamps, light emitting diodes and the like. As will be further developed below, the value of Fᵥ at the end of the incubation, measured as a single-point value or from an equation fitted to a time-series of measurements, is designated Fᵥ(1).

The sample is then incubated at a lower irradiance that is still high enough to allow for net photorepair and recovery of Fᵥ. For example, the sample can be incubated for a period of from about 30 minutes to about 240 minutes to PAR having an intensity of from about 10 μmol photons m⁻²s⁻¹ to about 50 μmol photons m⁻²s⁻¹. Preferably, the irradiance is dominated by wavelengths of from about 400 nm to about 700 nm and can be generated, for example, using a radiation sources as described in the preceding paragraph. The value of Fᵥ at the end of the incubation, measured as a single-point value or from an equation fitted to a time-series of measurements, is designated Fᵥ(2).

The photorepair index (PRI) is based on the ratio of Fᵥ in the treated sample to either the intial Fᵥ or the recovered Fᵥ in the untreated sample:

$$\begin{matrix}PRI = \frac{{[ {F}_{v} ( 2 ) - {F}_{v} ( 1 ) ]}^{Treated}}{{[ {F}_{v} ( 0 ) ]}^{Untreated}} & ( Eq . 1 ) \\ PRI = \frac{{[ {F}_{v} ( 2 ) - {F}_{v} ( 1 ) ]}^{Treated}}{{[ {F}_{v} ( 2 ) - {F}_{v} ( 1 ) ]}^{Untreated}} & ( Eq . 2 ) \\ PRI = \frac{{[ {F}_{v} ( 2 ) - {F}_{v} ( 1 ) ]}^{Treated}}{{{F}_{v} ( 1 )}^{Untreated}} . & ( Eq . 3 )\end{matrix}$$

where Fᵥ(1) and Fᵥ(2) are defined as described above, Untreated refers to control samples that are not exposed to treatments such as UVR, Treated refers to the sample post UV treatment, and Fᵥ(0) refers to the sample prior to any treatment.

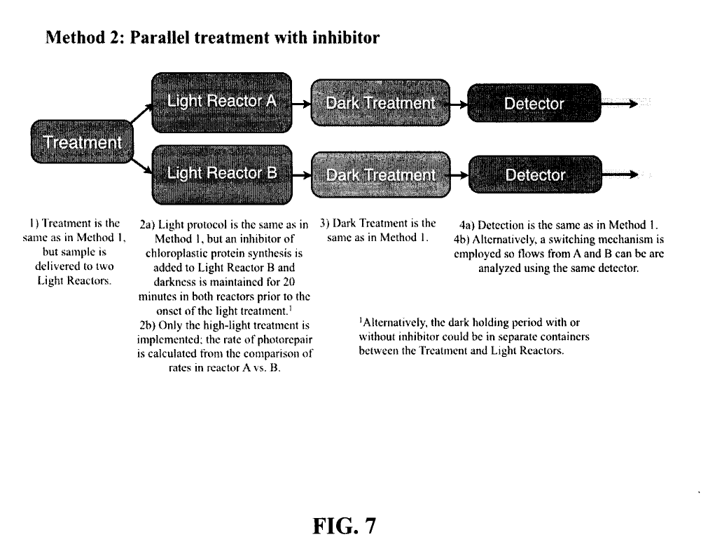

In an alternate embodiment of the invention, Fᵥ may also be measured during parallel incubations treated with and without a chloroplastic protein synthesis inhibitor (e.g., antibiotics such as streptomycin, lincomycin, azithromycin, etc.) and thereby incable of photorepair; the results can be used to validate interpretions of damage versus repair this is shown schematically in FIG. 7.

When determined with a protein synthesis inhibitor, it is again preferred that both the Untreated and UVR-treated (Treated) samples are subjected to the same assay protocol. Preferably, the protocol is as follows.

The sample is mixed and divided into two aliquots. One aliquot is treated with an appropriate dose of a protein synthesis inhibitor (e.g., 200-1000 g l⁻¹ lincomycin).

The untreated sample is a control. Both samples are incubated in darkness at assay temperature for a period of at least about 10 minutes, more preferably at least about 15 minutes, most preferably 20 minutes.

F_{v,Initial}. is measured on both untreated control and antibiotic-treated sub-samples prior to illumination.

Both sub-samples are then incubated at an irradiance high enough and for a period long enough to induce a prescribed or pre-determined reduction (e.g., 50%) in the untreated sub-sample—for example, using the above-described time periods and radiation intensities.

The photorepair index (PRI) is based on the difference in rates of decline of Fᵥ between the control and protein synthesis inhibitor-treated sub-samples. Preferably, the rates of decline are characterized by fitting to a first-order model, for example:

F_{v,t}=F_{v,Initial}·exp(−kt) (Eq. 4)

where F_{v,t} is Fᵥ at time t during high-light exposure, F_{v,Initial} is Fᵥ prior to high-light exposure, and k is a first-order rate constant that is evaluated for both the inhibited and uninhibited sub-samples (k_{Inhibited}, k_{Control}). Eq. 4 can be modified to include F_{v,∞}, a non-zero asymptotic value of Fᵥ:

F_{v,t}=F_{v,Initial}·exp(−kt)+F_{v,∞} (Eq. 5)

In either case, PRI is calculated as a function of the difference between inhibited and uninhibited rate constants for the Treated (e.g., with UVR) subsample vs. the Untreated sample:

$$PRI = \frac{{[ {F}_{v , Initial} • ( {k}_{Inhibited} - {k}_{Control} ) ]}^{Treated}}{{[ {F}_{v , Initial} • ( {k}_{Inhibited} - {k}_{Control} ) ]}^{Untreated}} \qquad ( Eq . 6 )$$

where k_{Inhibited} and k_{Control} are the rate constants for the sub-sample treated with the protein synthesis inhibitor and the control sub-sample, respectively, and where Treated refers to the sample post-UVR treatment, and Untreated refers to the sample prior to any treatment.

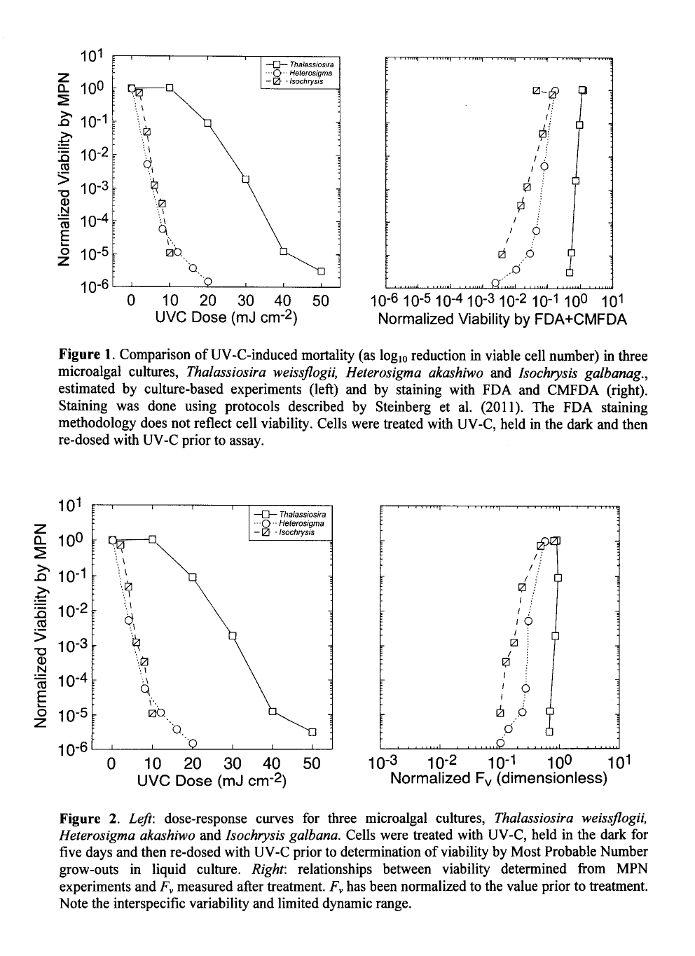

The photorepair index is related to the probability of survivorship as determined through experiments on microorganisms subjected to UVC and assayed for both PRI and the reduction in viability as determined through culture-based experiments (e.g., Most Probable Numbers, MPN, FIG. 1-3).

Preferred embodiments of the present application will be illustrated with reference to the following examples, which are not intended to construe or limit the scope of the present invention:

### EXAMPLE 1

Cultures of marine microalgae were used in the Examples. These were obtained from the Provasoli-Guillard National Center for Marine Algae (East Boothbay, Me., USA) and maintained at low optical density at 18° C. on a 12:12 L:D cycle.

Illumination was provided by cool-white fluorescent bulbs at an intensity of 80 μmol photons m⁻²s⁻¹ PAR.

Cultures were maintained in nutrient-replete balanced growth at constant density by daily dilution with fresh medium (Maclntyre and Cullen 2005). Cultures were monitored daily for dark-acclimated chlorophyll a fluorescence (Brand et al., 1981) using a 10-AU fluorometer (Turner Designs, San Jose, Calif., USA) and a FIRe fluorometer (Satlantic, Halifax, NS, Canada). Both fluorometers were blanked daily and fluorescence was normalized to a 200 μM rhodamine standard.

Daily specific growth rates (μ, (d⁻¹) were calculated from the dilution-corrected change in fluorescence over the preceding 24 h assuming exponential growth (Maclntyre and Cullen 2005). The single-turnover fluorescence induction curve measured with the FIRe was fitted using the to MATLAB routine Fireworx 1.0.4 (Barnett) to estimate minimum and maximum fluorescence (F₀ and Fₘ, Arb.), variable fluorescence (Fᵥ=Fₘ−F₀, Arb.), and the quantum yield of Photosystem II electron transport (Fᵥ/Fₘ, dimensionless). Cultures were considered to be in balanced growth when the coefficients of variation (C.V.) for μ and Fᵥ/Fₘ were <10% for a minimum of 10 generations.

Cultures in balanced growth were subjected to defined doses of UV-C radiation delivered by a conventional low-pressure collimated beam source (Bolton and Linden 2003). Cultures were irradiated in 50-ml aliquots in a reaction vessel (50 mm diameter, 25 mm depth) centered under the UV beam. Cultures were stirred (approx. 60 r.p.m.) with a miniature magnetic stir-bar during dosage to ensure homogenous application of the dose.

The intensity of the UV beam was measured with a NST-traceable ILT1700 radiometer (International Light Technologies, Peabody, Mass., USA). Homogeneity of exposure over the surface of the reaction vessel was verified as being <5% (C.V.) by measuring beam intensity at 5-mm intervals over perpendicular axes aligned with the center of the reaction vessel. Beam attenuation through the culture at 254 nm was measured with a UV254 Series ‘P’ meter RealTech, Whitby, ON). The mean dosage in the reaction vessel was calculated from the incident intensity and the attenuation at 254 nm, by application of the Lambert-Beer law. The dosage (mJ cm⁻²) was then set by calculating the appropriate duration of exposure, given that dosage is the product of the mean intensity in the reaction vessel (mW cm⁻²) and the duration of exposure(s).

A photorepair index was calculated from the recovery of Fᵥ at low irradiance following application of a photoinhibitory PAR light regime—see FIG. 4. An exponentially-growing culture was divided into two aliquots. One, the Untreated, sample, was assayed immediately; the second Treated sample was irradiated with UV-C before assay.

The two samples were otherwise subjected to identical assay conditions. Each was dark-acclimated for a minimum of 20 minutes to allow photochemical quenching to be restored and short-lived fluorescence quenching to relax. A subsample was taken at the end of this period and Fᵥ was measured using the FIRe fluorometer. The remainder of the sample was then incubated at an irradiance of 550 μmol photons m⁻²s⁻¹ of photosynthetically active radiation (PAR, 400 nm-700 nm) for 60 minutes.

The sample was held at growth temperature in a water-cooled manifold illuminated by a programmable warm-white LED array (Photon Systems International, Brno, Czech Republic). Sub-samples were removed at 10-min intervals and dark-acclimated for a minimum of 20 minutes prior to determination of Fᵥ. These are designated as the “High-Light” samples in FIG. 4.

Following the high-light exposure, the remaining sample was incubated at growth temperature at an irradiance of 20 μmol photons m⁻²s⁻¹ of PAR by the same LED array. Sub-samples were removed at 15-minute intervals and dark-acclimated for a minimum of 20 minutes for determination of Fᵥ. These are designated as the “Low-Light” samples in FIG. 4.

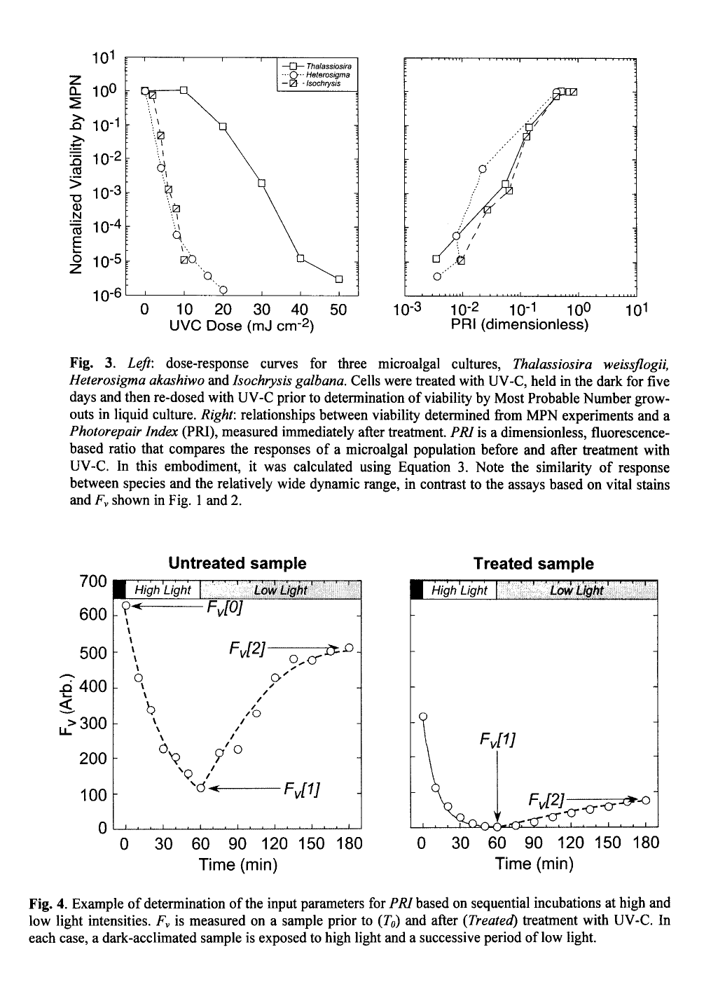

The input parameters for permutations of the PRI were derived by fitting the kinetic variations in Fᵥ over time in the two different regimes and for each of the Untreated and Treated samples and the results are shown in FIG. 4 (results are shown in FIG. 3 for several different treatments).

### EXAMPLE 2

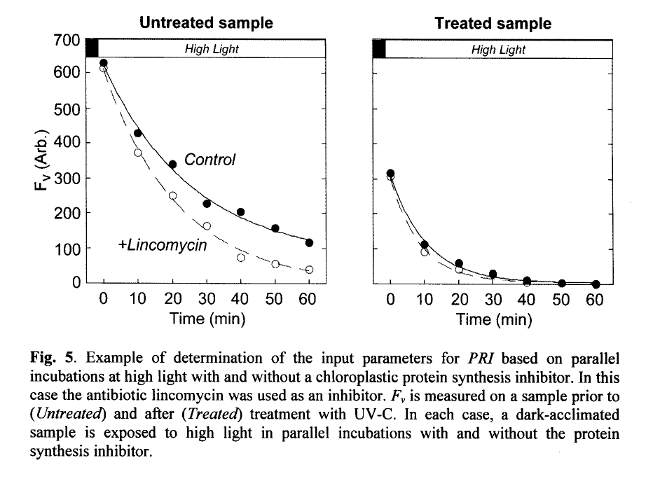

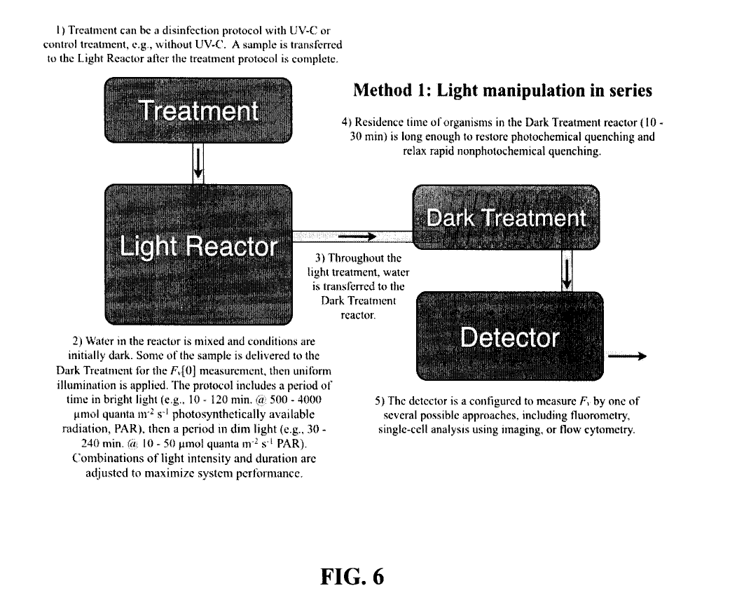

In this example, the photorepair index was calculated from the differential loss of Fᵥ during application of a photoinhibitory light regime with and without the antibiotic lincomycin, an inhibitor of chloroplastic protein synthesis—see FIG. 5.

An exponentially-growing culture was divided into two aliquots. One, the Untreated sample, was assayed immediately; the second Treated sample was irradiated with UV-C before assay.

Both the Untreated and the Treated samples were then subdivided into control and antibiotic-treated subsamples. The control samples were assayed without further amendment. The antibiotic-treated samples were treated with an aqueous solution of lincomycin to a final concentration of 500 μg ml⁻¹ and incubated at growth temperature in the dark for 10 minutes to allow uptake of the antibiotic.

Subsequently, all four samples were subjected to identical assay conditions. Each was first dark-acclimated for a minimum of 20 min to allow short-lived fluorescence quenching to relax. A subsample was taken at the end of this period and Fᵥ was measured using the FIRe fluorometer. The remainder of the sample was then incubated at an irradiance of 550 μmol photons m⁻² s⁻¹ of Photosynthetically Active Radiation (PAR, 400-700 nm) for 60 minutes. The sample was held at growth temperature in a water-cooled manifold illuminated by a programmable warm-white LED array (Photon Systems International, Brno, Czech Republic). Sub-samples were removed at 10-min intervals and dark-acclimated for a minimum of 20 min for determination of Fᵥ. These are designated as the “High-Light” samples in FIG. 5.

The input parameters for permutations of the PRI were derived by fitting the kinetic variations in Fᵥ over time in both the control and antibiotic-treated subsamples of each of the Untreated and Treated samples.

### EXAMPLE 3

Viability of the cultures subsequent to treatment with UV-C was assessed by the Most Probable Number (MPN) assay (Cochran 1950; Blodgett 2005a,b).

Thus, the cultures were diluted in 3 log-interval series (e.g. 10⁻¹, 10⁻² and 10⁻³) with fresh growth medium. The appropriate dilution range for any UV-C dose was determined in preliminary, range-finding experiments.

For each culture, five replicates of each dilution were then incubated at an irradiance and temperature optimal for growth (typically 160 μmol photons m⁻²s⁻¹ of PAR and 22° C.) and monitored for growth every 48 h using the 10-AU fluorometer. Tubes were scored positive for growth if fluorescence increased by an order of magnitude above the limit of quantitation (Anderson 1989) or the initial fluorescence reading, whichever was higher.

The Most Probable Number of viable cells was then obtained from look-up tables (Blodgett 2010) and converted to a concentration from the volume of culture in each tube and the range of dilutions used. The concentrations of viable cells obtained by the MPN analyses were used to construct dose-response curves for UV exposure and for comparison with the PRI—see FIG. 3. As can be seen in FIG. 3, the similarity of response between species and the relatively wide dynamic range is superior to the assays based on vital-stain and Fᵥ shown in FIGS. 1 and 2.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. For example, while a preferred embodiment of the present invention relates to the use of fluorescence in an assay for loss of organism (preferably microorganism) viability in an aqueous liquid, it is possible to adapt this preferred embodiment to the use of fluorescence in an assay for loss of organism (preferably microorganism) viability in other than an aqueous liquid—e.g., organisms (preferably microorganisms) that have been isolated on a filter or otherwise removed from the medium in which they typically exist. Thus, it is possible to modify the schematic illustrated in FIG. 6 to include filter element or other organism (preferably microorganism) isolating element prior to the Dark Treatment or Detector elements. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

### LIST OF DOCUMENTS CITED IN SPECIFICATION

(1) Adir, N., H. Zer, S. Shochat and I. Ohad (2005). Photoinhibition—a historical perspective. Discoveries in Photosynthesis. Govindjee, J. T. Beatty, H. Gest and J. F. Allen. Dordrecht, The Netherlands, Springer: 931-958.

(2) Agusti, S. and M. C. Sánchez (2002). Cell viability in natural phytoplankton communities quantified by a membrane permeability probe. Limnol. Oceanogr. 47(3): 818-828.

(3) Anderson, D. J. (1989). “Determination of the lower limit of detection.” Clin. Chem. 35(10): 2152-2153.

(4) Blodgett, R. J. (2005a). “Serial dilution with a confirmation step.” Food Microbiol. 22(6): 547-552.

(5) Blodgett, R. J. (2005b). “Upper and lower bounds for a serial dilution test.” J. AOAC Int. 88(4): 1227-1230.

(6) Blodgett, R. J. (2010). Appendix 2: Most Probable Number from Serial Dilutions. Bacteriological Analytical Manual, US Food and Drug Administration.

(7) Bolton, J. R. and K. G. Linden (2003). “Standardization of methods for fluence (UV dose) determination in bench-scale UV experiments.” J. Environ. Eng.-ASCE 129(3): 209-215.

(8) Bouchard, J. N., D. A. Campbell and S. Roy (2005). Effects of UV-B radiation on the D1 protein repair cycle of natural phytoplankton communities from three latitudes (Canada, Brazil, and Argentina). J. Phycol. 41: 273-286.

(9) Brand, L. E., R. R. L. Guillard, and L.S. Murphy (1981). “A method for the rapid and precise determination of acclimated phytoplankton reproduction rates.” J. Plankton Res. 3: 193-201.

(10) Bouchard, J. N., M. L. Longhi, S. Roy, D. A. Campbell and G. Ferreyra (2008). Interaction of nitrogen status and UVB sensitivity in a temperate phytoplankton assemblage. J. Exp. Mar. Biol. Ecol. 359: 67-76.

(11) Campbell, D., V. Hurry, A. K. Clarke, P. Gustafsson and G. Öquist (1998). Chlorophyll fluorescence analysis of cyanobacterial photosynthesis and acclimation. Microbiology and Molecular Biology Reviews 62: 667-683.

(12) Cochran, W. G. (1950). “Estimation of bacterial densities by means of the most probable number.” Biometrics 6(2): 105-116.

(13) Cullen, J. J., M. Zhu and D. C. Pierson (1986). A technique to assess the harmful effects of sampling and containment for determination of primary production. Limnol. Oceanogr. 31: 1364-1373.

(14) Dorsey, J., C. M. Yentsch, S. Mayo and C. McKenna (1989). Rapid analytical technique for the assessment of cell metabolic activity in marine microalgae. Cytometry 10: 622-628.

(15) ETV (2010). Generic protocol for the verification of ballast water treatment technology. Program, NSF International for USEPA Environmental Technology Verification Program, Ann Arbor, Mich.

(16) Gorbunov, M. Y. and P. G. Falkowski. Fluorescence induction and relaxation (FIRe) technique and instrumentation for monitoring photosynthetic processes and primary production in aquatic ecosystems. Fundamental Aspects to Global Perspectives, Bruce, D., and A. van der zo Est, Eds. Allen Press, Montreal. P. 1029-1031.

(17) First, M. R. and L. A. Drake (2013a). Approaches for determining the effects of UV radiation on microorganisms in ballast water. Management of Biological Invasions 4: 87-99.

(18) First, M. R. and L. A. Drake (2013b). Life after treatment: detecting living microorganisms following exposure to UV light and chlorine dioxide. J Appl Phycol DOI 10.1007/s10811-013-0049-9.

(19) Garvey, M., B. Moriceau and U. Passow (2007). Applicability of the FDA assay to determine the viability of marine phytoplankton under different environmental conditions. Marine Ecology-Progress Series 352: 17-26.

(20) Genty, B., J.-M. Briantais and N. R. Baker (1989). The relationship between the quantum yield of photosynthetic electron transport and quenching of chlorophyll fluorescence. Biochem. Biophys. Acta 990: 87-92.

(21) Gieskes, W. W. C. and A. G. J. Buma (1997). UV damage to plant life in a photobiologically dynamic environment: The case of marine phytoplankton. Plant Ecol. 128(1-2): 16-25.

(22) Gilbert, F., F. Galgani and Y. Cadiou (1992). Rapid assessment of metabolic-activity in marine microalgae—application in ecotoxicological tests and evaluation of water-quality. Mar. Biol. 112(2): 199-205.

(23) Key, T., A. McCarthy, D. A. Campbell, C. Six, S. Roy and Z. V. Finkel (2010). Cell size trade-offs govern light exploitation strategies in marine phytoplankton. Environmental Microbiology 12: 95-104.

(24) Liebich, V., P. P. Stehouwer and M. Veldhuis (2012). Re-growth of potential invasive phytoplankton following UV-based ballast water treatment. Aquat. Invasions 7(1): 29-36.

(25) Maclntyre, H. L. and J. J. Cullen (2005). Using cultures to investigate the physiological ecology of microalgae. Algal Culture Techniques. R. A. Andersen. New York, Academic Press: 287-326.

(26) Murphy, A. and T. Cowles (1997). Effects of darkness on multi-excitation in vivo fluorescence and survival in a marine diatom. Limnol. Oceanogr. 42: 1444-1453.

(27) Neidhardt, J., J. R. Benemann, L. Zhang and A. Melis (1998). Photosystem-II repair and chloroplast recovery from irradiance stress: relationship between chronic photoinhibition, light-harvesting chlorophyll antenna size and photosynthetic productivity in Dunaliella salina (green algae). Photosynth. Res. 56: 175-184.

(28) Onji, M., T. Sawabe and Y. Ezura (2000). An evaluation of viable staining dyes suitable for marine phytoplankton. Bull Fac Fish Hokkaido Univ 51: 151-158.

(29) Peperzak, L. and C. P. D. Brussaard (2011). Flow cytometric applicability of fluorescent vitality probes on phytoplankton. J. Phycol. 47(3): 692-702.

(30) Schreiber, U., H. Hormann, C. Neubauer and C. Klughammer (1995). Assessment of photosystem II photochemical quantum yield by chlorophyll fluorescence quenching analysis. Aust. J. Plant Physiol. 22: 209-220.

(31) Selvin, R., B. Requera, I. Bravo and C. M. Yentsch (1988). Use of fluorescein diacetate (FDA) as a single-cell probe of metabolic activity in dinoflagellate cultures. Bio. Oceanogr. 6: 505-511.

(32) Sinha, R. P. and D.-P. Hader (2002). UV-induced DNA damage and repair: a review. Photochemical & Photobiological Sciences 1(4): 225-236.

(33) Steinberg, M. K., E. J. Lemieux and L. A. Drake (2011). Determining the viability of marine protists using a combination of vital, fluorescent stains. Mar. Biol. 158: 1431-1437.

(34) Throndsen, J. (1978), The dilution-culture method, in Phytoplankton Manual, edited by A. Sournia, pp. 218-224, UNESCO, Paris.

(35) Vasilikiotis, C. and A. Melis (1994). Photosystem II reaction center damage and repair cycle: chloroplast acclimation strategy to irradiance stress. Proc. Nat. Acad. Sci 91: 7222-7226.

(36) Vincent, W. F. (1980). Mechanisms of rapid photosynthetic adaptation in natural phytoplankton communities. II. Changes in photochemical capacity as measured by DCMU-induced chlorophyll fluorescence. J. Phycol. 16: 568-577.

(37) Xiong, F. (2001). Evidence that UV-B tolerance of the photosynthetic apparatus in microalgae is related to the D1-turnover mediated repair cycle in vivo. J. Plant Phys. 158: 285-294.

1-13. (canceled)

14. A method for assaying for loss of viability of an organism in an aqueous liquid after the organism has been exposed to a stressor, the method comprising the step of assessing the ability of the organism to undergo photorepair.

15. The method defined in claim 14, wherein the stressor is selected from the group consisting of: exposure to a chemical, exposure to mechanical energy, thermal shock, dark storage, and any combination thereof.

16-17. (canceled)

18. The method defined in claim 14, wherein the stressor is ultraviolet radiation having a wavelength in the range of from about 100 nm to about 280 nm.

19. (canceled)

20. The method defined in claim 14, wherein the assessing step comprises conducting a variable fluorescence test on the organism.

21. The method defined in claim 14, wherein the organism is a microorganism.

22-23. (canceled)

24. A method for assaying for loss of viability of an organism in an aqueous liquid after the organism has been exposed to a stressor, the method comprising the step of correlating the photorepair index for the organism in the aqueous liquid to survivorship of the organism after the organism has been exposed to the stressor.

25-33. (canceled)

34. A method for assaying for loss of viability of an organism comprised in an aqueous liquid after the organism has been exposed to a stressor, the method comprising the steps of:
(a) measuring the variable fluorescence (Fᵥ) of an untreated sample of the organism prior to exposure to the stressor;
(b) measuring the variable fluorescence (Fᵥ) of a treated sample of the organism after exposure to the stressor;
(c) calculating a photorepair index using the measurements obtained in Steps (a) and (b); and
(d) correlating the photorepair index calculated in Step (c) to a normalized viability for the organism.

35. The method defined in claim 34, wherein Step (a) comprises one or more of the following:
measuring the variable fluorescence (Fᵥ) of the untreated sample of the organism prior to exposure to radiation to obtain Fᵥ(0)^{Untreated};
(ii) incubating the untreated sample in the presence of radiation at a first intensity to induce a prescribed reduction in Fᵥ from Fᵥ(0)^{Untreated};
(iii) measuring the variable fluorescence (Fᵥ) of the untreated sample after Step (ii) to obtain Fᵥ(1)^{Untreated};
(iv) incubating the untreated sample in presence of radiation at a second intensity to induce photorepair and an increase in Fᵥ from Fᵥ(1)^{Untreated}; and
(v) measuring the variable fluorescence of the untreated sample after Step (iv) to obtain Fᵥ(₂)^{Untreated}.

36. The method defined in claim 35, wherein Step (ii) is conducted for a period of from about 10 minutes to about 120 minutes.

37. The method defined in claim 35, wherein the first intensity is in the range of from about 2 μmol photons m⁻²s⁻¹ to about 4000 μmol photons m⁻²s⁻¹.

38. The method defined in claim 35, wherein Step (ii) comprises incubating the untreated sample in presence of photosynthetically active radiation.

39. The method defined in claim 35, wherein Step (ii) comprises incubating the untreated sample in presence of photosynthetically active radiation having one or more wavelengths substantially within the range of from about 400 nm to about 700 nm.

40. (canceled)

41. The method defined in claim 35, wherein the prescribed reduction in Fᵥ in Step (ii) is in the range of from about 30% to 80%.

42. (canceled)

43. The method defined in claim 35, wherein Step (iv) is conducted for a period of from about 30 minutes to about 240 minutes.

44. The method defined in claim 35, wherein the second intensity is in the range of from about 1 μmol photons m⁻²s⁻¹ to about 50 μmol photons m⁻²s⁻¹.

45-46. (canceled)

47. The method defined in claim 35, wherein Step (iv) comprises incubating the untreated sample in presence of photosynthetically active radiation having one or more wavelengths substantially within the range of from about 400 nm to about 700 nm.

48. (canceled)

49. The method defined in claim 35, wherein Step (b) comprises one or more of the following:
(i) incubating the treated sample in presence of photosynthetically active radiation at a first intensity to induce a prescribed reduction in Fᵥ from Fᵥ(0)^{Untreated};
(ii) measuring the variable fluorescence of the treated sample after Step (ii) to obtain Fᵥ(1)^{Treated};
(iii) incubating the untreated sample in presence of radiation at a second intensity to induce photorepair and an increase in Fᵥ from Fᵥ(1)^{Treated}; and
(iv) measuring the variable fluorescence of the treated sample after Step (iii) to obtain Fᵥ(₂)^{Treated}.

50-52. (canceled)

53. The method defined in claim 49, wherein Step (i) comprises incubating the untreated sample in presence of photosynthetically active radiation having one or more wavelengths substantially within the range of from about 400 nm to about 700 nm.

54-58. (canceled)

59. The method defined in claim 49, wherein substantially the same radiation is used Step (i) and Step (iii).

60. The method defined in claim 35, wherein Step (c) comprises calculating the photorepair index using one of the following equations:
$$\begin{matrix}PRI = \frac{{[ {F}_{v} ( 2 ) - {F}_{v} ( 1 ) ]}^{Treated}}{{[ {F}_{v} ( 0 ) ]}^{Untreated}} & ( Eq . 1 ) \\ PRI = \frac{{[ {F}_{v} ( 2 ) - {F}_{v} ( 1 ) ]}^{Treated}}{{[ {F}_{v} ( 2 ) - {F}_{v} ( 1 ) ]}^{Untreated}} & ( Eq . 2 ) \\ PRI = \frac{{[ {F}_{v} ( 2 ) - {F}_{v} ( 1 ) ]}^{Treated}}{{{F}_{v} ( 1 )}^{Untreated}} . & ( Eq . 3 )\end{matrix}$$

61-65. (canceled)
